(19) United States
(12) Patent Application Publication
(10) Pub. No.: US 2008/0097288 A1
(43) Pub. Date: Apr. 24, 2008

(54) Disposable blood glucose sensor with internal pump

(76) Inventors: Paul D. Levin, Scotts Valley, CA (US); Henry Grage, Alpharetta, GA (US)

Correspondence Address: Bruce H. Johnsonbaugh;Eckhoff & Hoppe, Suite 2800, 101 Montgomery Street, San Francisco, CA, 94104, US

(21) Appl. No.: 11/891,701

(22) Filed: Aug. 13, 2007

Related U.S. Application Data
(63) Continuation-in-part of application No. 11/827,889, filed on Jul. 13, 2007.
(60) Provisional application No. 60/853,413, filed on Oct. 20, 2006.

(51) Int. Cl. A61M 31/00; A61B 5/05

(52) U.S. Cl. 604/66; 600/347; 604/508

(57) ABSTRACT

An apparatus and method are disclosed for automatically and periodically measuring the level of a patient's blood glucose when a patient has a catheter in a blood vessel. A wearable, disposable test unit is attached by air, fluid and electric lines to a bedside monitor. The test unit has means for measuring blood glucose. A chamber in the unit contains a pneumatic pump which draws blood into the device and then expels all but a small residual portion of blood back into the patient's blood vessel catheter. A testing area is provided adjacent the pumping chamber. A test is done either when the pumping chamber is filled with blood, or after the pumping chamber is emptied. The test cycle is repeated about every 60 seconds. Provision is made to automatically calibrate the device every few hours or whenever calibration is required.

### CROSS-REFERENCE TO RELATED APPLICATIONS

This application is a continuation-in-part of U.S. application Ser. No. 11/827,889, filed Jul. 13, 2007.

This application claims the benefit of and priority from U.S. provisional application Ser. No. 60/853,413 filed Oct. 20, 2006.

### BACKGROUND AND SUMMARY OF THE INVENTION

This invention relates to blood glucose testing in critically ill patients. Attempts have been made in the past to automatically monitor blood glucose from a patient's IV line. Generally these systems have used a peristaltic pump to reverse the direction of flow in an infusion line so that blood could be pulled out of the patient's circulation at intervals, analyzed, and then re-infused into the patient. Examples of such device are described in U.S. Pat. No. 3,910,256 to Clark, U.S. Pat. No. 4,573,968 to Parker and U.S. Pat. Nos. 5,165,406, 5,758,643 and 5,947,911 to Wong and associates.

A problem with prior systems for testing blood glucose, as in the inventions of Wong and others, is the tendency for blood and infusion fluid to mix when IV infusion fluid is moved back and forth by a peristaltic pump. Since blood is quite viscous, and clear infusion fluid is not, blood tends to move at a slower rate then clear fluid, sticking to the inner walls of the IV tubing and thereby causing blood and infusion fluid to mix rapidly as the two travel back and forth through the plastic tubing. Generally within a few seconds blood and fluid are intermixed over a distance of 10-20 cm making it difficult to obtain a pure blood sample for analysis. After testing the sample must be completely cleared from the test area. Blood tends to adhere to the inner wall of the tubing and test chamber making it necessary to use a fairly large volume of fluid to completely clear the sample. Typically a minimum of 4-5 ml of infusion fluid is needed for complete clearance. This volume of fluid is unacceptable if infused every few minutes as it could overload the patient's circulation.

The system as described by Wong and others is presently being marketed by International Biomedical of Austin, Tex. for the monitoring of blood gases. In order to avoid the fluid overload problem, particularly in pediatric patients, or adults in heart failure, a system of tubing and valves has been added for disposal of fluid waste, specifically the wash fluid needed after every test to clean the system of the previous sample. The additional valves are needed to redirect the fluid path into a waste bag, which must be periodically discarded. The system is rather complex and requires about 15 minutes of set-up time prior to use.

A second disadvantage of allowing blood and infusion fluid to mix is uncertainty of the purity of the sample. To overcome this problem, an optical system can be used to test whether only pure blood is in the test area. Such a system is described in U.S. Pat. No. 7,162,290 to Levin. Optics add complexity and cost, however, to a disposable device and if possible are best avoided.

The present invention avoids the above-mentioned problems by automatically withdrawing and returning a blood sample without allowing contact between the sample and infusion fluid, except during an occasional calibration. The disposable sensor is small and unobtrusive and the cost of the bedside monitor is lowered by eliminating the extra valves required to divert fluid into a waste bag as used in prior art systems.

The system to be described uses a bedside monitor in combination with a small wearable, disposable sensor. The sensing unit carries within it a pumping mechanism which can draw in about 2 ML of blood from a patient's intravascular catheter into the sensor and then expel most of the withdrawn blood back into the patient's circulation. Blood in the testing area may be tested either: a) when the withdrawn blood initially fills the testing area, or b) after most of the blood has been returned to the patient's body, but wherein the small amount of residual blood in the testing area of the sensor may be tested for blood glucose. Each cycle of drawing in and then returning a blood sample is repeated every 60 seconds, which is too short a time to allow the formation of clots. Since the blood sample is isolated from and never mixes with infusion fluid, as in prior art systems, there is no dilution of the blood being tested and the sample is always pure. When using the present invention, it is essential that the patient's intra-vascular catheter be inside a dedicated blood vessel. If a multi-lumen central venous catheter is used for vascular access, the proximal lumen should be selected for drawing blood samples. The proximal lumen will be up-stream of the remaining lumens and blood samples will therefore be unaffected by infusion fluids introduced through them.

The disposable sensor of the present invention is designed to be worn by the patient either on the chest for monitoring from a central venous catheter or on an extremity for monitoring from a peripheral artery or vein. In one embodiment of the sensor, a channel carries a working electrode and a counter electrode. The working electrode uses glucose oxidase. As is well known in the art, glucose oxidase on a platinized surface will cause the production of hydrogen peroxide in the presence of glucose, water and oxygen. Hydrogen peroxide is then immediately broken down by the catalytic action of platinum to cause the release of one electron for each molecule of glucose that participated in the first reaction. Amperometric measurement of electron flow between the working and counter electrodes is used to determine the glucose concentration of the fluid inside the sensor. Non-enzymatic glucose sensors can also be adapted to the present invention. For example, U.S. Pat. Nos. 6,653,141 and 6,627,177 and U.S. applications 2006.008.3688 and 2004.002.8612 disclose chemical compounds which can be applied to the end of an optical fiber to measure blood glucose. An optical fiber sensor can be used in combination with the present invention.

Two methods of drawing blood into the sensor are described. Both variations are able to cyclically draw about 2 ML of blood into the sensor, and then quickly expel it back into the patient. Approximately 200 micro liters of residual blood can be tested by the sensor's electrode, or a test can be done when the device is filled with blood. In the first embodiment of the invention, a balloon connected to a catheter is used to pump blood in and out of the sensor at about one minute intervals. In the second embodiment of the present invention, an elastic membrane inside the sensor is drawn up into a dome and the negative pressure created under the membrane draws in blood from the patient's intravascular catheter. The first embodiment is preferred because of its slightly smaller size and ease of manufacture.

Air, fluid and electric lines to the wearable disposable sensor connect to a bedside monitor. A motor driven precision syringe inside the monitor is connected by an air line to the pumping mechanism inside the sensor carrying either embodiment of the invention.

The bedside monitor has a digital display to show the most recent value for the patient's blood glucose. Trends can also be displayed. An alarm inside the monitor alerts caregivers to any excursion of a blood glucose outside the pre-set alarm limits. In addition to the motor driven syringe to pressurize or depressurize the air line, the monitor carries a peristaltic pump, which can move calibration fluid through the multi-lumen tube connecting the monitor to the wearable sensor.

A primary object of the invention is to provide a blood glucose testing system which introduces only small amounts of additional fluid into the patient's circulation.

A further object of the invention is to provide a pure blood sample in the test area without the use of special optics.

Another object of the invention is to produce a system which can be used with either a peripheral or a central venous catheter or an arterial line.

A further object of the invention is to provide a system which is automatically self-calibrating.

Another object of the invention is to provide a low profile blood glucose sensor which can be comfortably worn on either the chest or extremities.

Other objects and advantages of the invention will become apparent from the following description and drawings.

### BRIEF DESCRIPTION OF THE DRAWINGS

FIG. 1 is an overall view of the present invention used in the environment of an intensive care unit;

FIG. 2 is a perspective view of a disposable testing unit of the present invention attached to a patient's forearm;

FIG. 3 is a close-up view of the bedside monitor showing the various leads and attachments;

FIG. 4 is a cross-sectional longitudinal view of the balloon pump version, with an enzyme electrode, enlarged to 2× scale for clarity;

FIG. 5 is the view seen in FIG. 4 reduced to its actual size;

FIG. 6 is a longitudinal midline vertical view of the disposable sensor showing the four different plastic parts, which are joined together to form the body of the device;

FIG. 7 is a cross-sectional view through the proximal part of the device containing the balloon pump and valve;

FIG. 8 is a cross-sectional view through the distal part of the sensor showing the working and counter electrodes;

FIG. 9 is a cross sectional longitudinal view of another version of the sensor showing how an optical fiber can be used to measure blood glucose;

FIGS. 10-12 show the sequence of steps in testing for blood glucose using an enzyme electrode;

FIGS. 13 and 14 show the sequence of steps in calibration of the device;

FIG. 15 is a cross-sectional vertical view of the elastic membrane version;

FIG. 16 is a cross-sectional horizontal view of the sensor of FIG. 15 along the line 16-16 of FIG. 15;

FIG. 17 is a cross-sectional horizontal view through the line 17-17 of FIG. 15; and

FIGS. 18-21 show the sequence of steps taken in the measurement of blood glucose using the elastic membrane version (FIGS. 15-17) of the present invention.

### DETAILED DESCRIPTION OF THE DRAWINGS

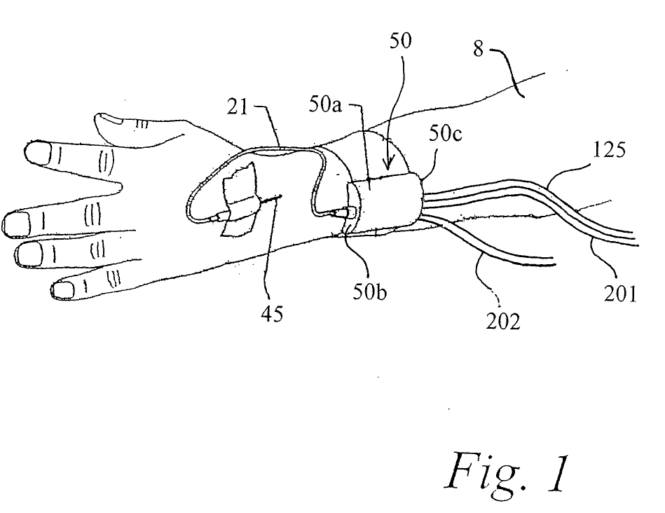

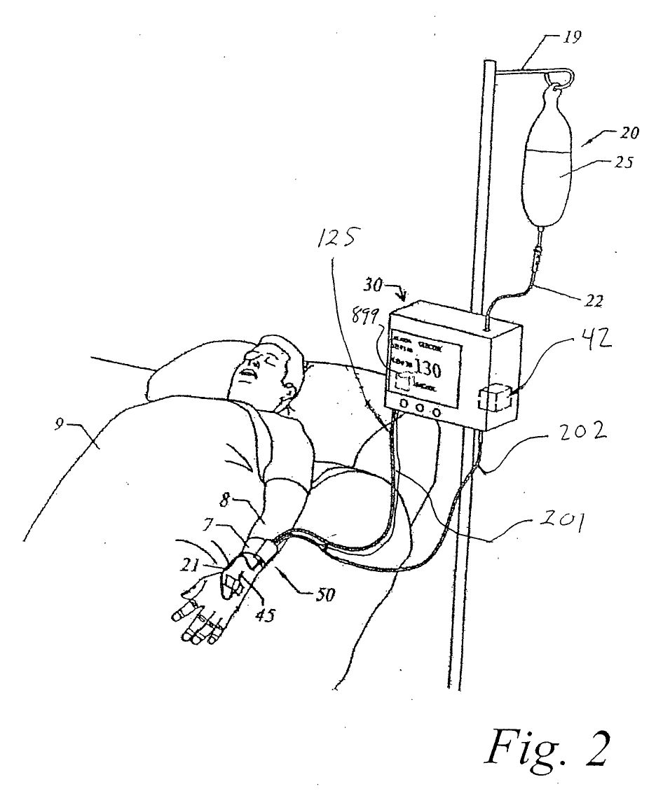

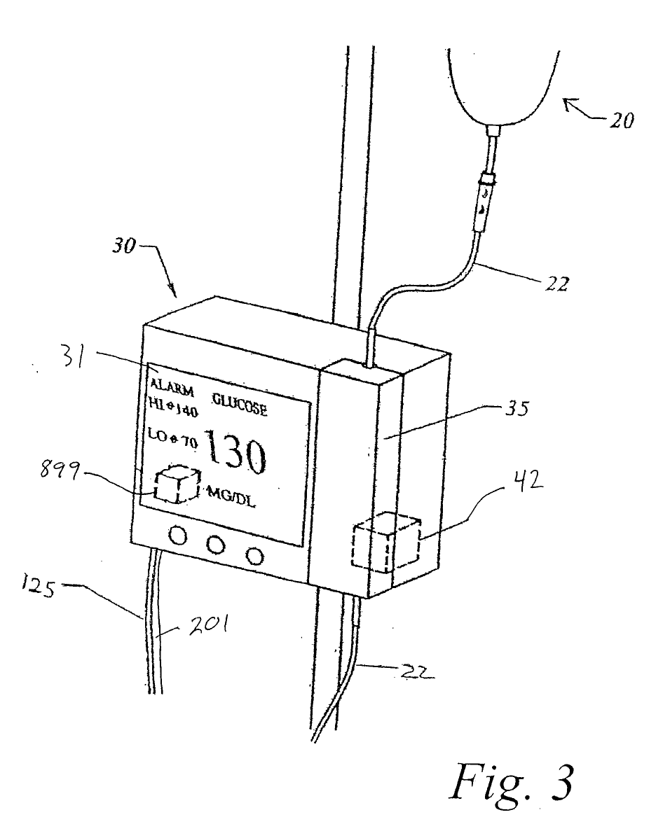

FIGS. 1, 2 and 3 illustrate the overall environment of the invention. A hospitalized patient 9 typically in an ICU unit is shown with a disposable testing unit 50 of the present invention attached to forearm 8 by a Coban elastic band. A catheter 45 is shown inserted into a patient blood vessel on the back of the patient's hand. An infusion line 21 connects catheter 45 to testing unit 50. A source of calibration fluid is shown as IV bag 20 suspended from a support 19 as is known in the art. Calibration fluid 25 stored in infusion bag 20 passes downwardly through an infusion line 202 and through a peristaltic pump 42 carried in monitor housing 30. The infusion line 202 continues downwardly from peristaltic pump 42 and enters the test unit 50. The peristaltic pump 42 operates only in the forward mode. Calibration fluid 25 from source 20 is intermittently pumped through infusion line 202 and through testing unit 50 and then through infusion line 21 attached to IV catheter 45. Electrical power is fed to testing unit 50 by line 125 extending from monitor 30. Air line 201 extends from monitor 30 to test unit 50. Air, fluid and electric lines are shown separately for clarity but in reality are in a multi-lumen tube running between the sensor and the bedside monitor. A motor driven syringe 899 carried in monitor 30 causes either positive or negative pressure in air line 201 and acts as an actuation means to cause expansion or contraction of balloon 60 or motion of elastic membrane 140 as described in detail below. It is significant to note that the infusion fluid used in the calibration procedure only enters the sensing unit during calibration. Calibration is done about every 2 hours or only about one time per 100 blood testing cycles.

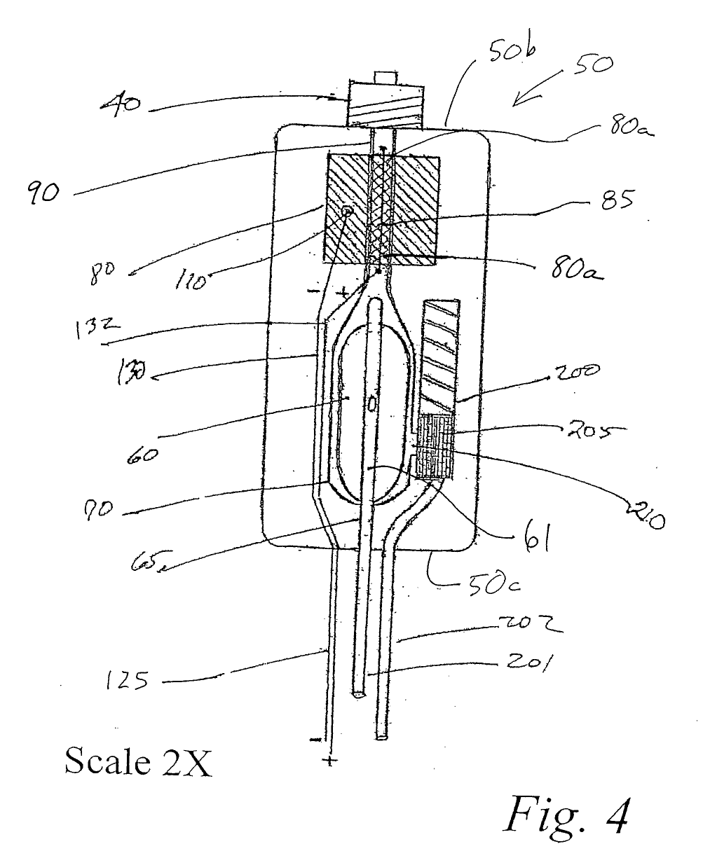

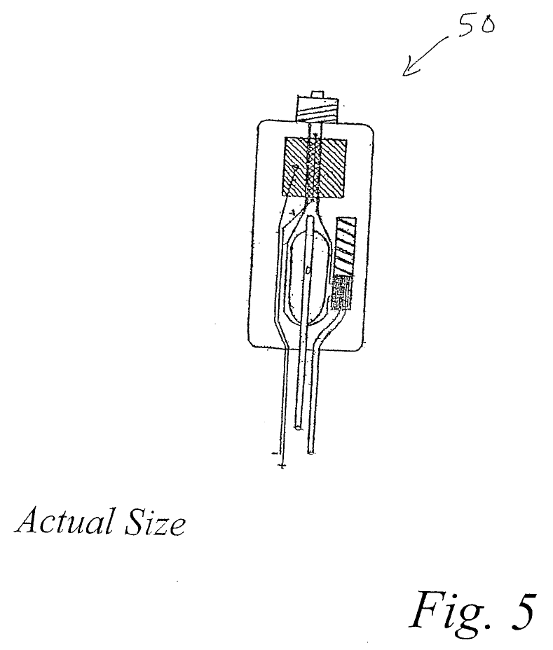

FIG. 4 and all other drawings except FIG. 5 are drawn 2× scale for clarity. FIG. 4 is a horizontal cross-sectional view through the central plane of the disposable test unit 50 in the first embodiment of the invention. The unit has a body 50a that is approximately 57 mm long and 30 mm wide and 15 mm high. Body 50a has a proximal end 50b and distal end 50c. A balloon 60 with actuating catheter 61 is fixed to air line 201 and positioned in a balloon (or pumping) chamber 70. A balloon and catheter customized for this application can be made by the Tech Device Corporation of Watertown, Mass. The balloon chamber is in continuity with a channel or passageway 90, which is approximately 2.5×2.5 mm in cross-sectional dimension. A luer fitting 40 is fixed to this channel enabling a catheter attached to the luer fitting to pass blood or fluid between the patient and the test or sensing unit 50. A rectangular piece of platinized kapton 80, 14×14 mm and 125 microns in thickness forms the floor of channel 90, although only the central part 80a of the kapton film comes in contact with blood or fluid. Electrical wires 130 and 132 contact the working and counter electrodes 80a and 85 of the glucose sensor and carry electric changes to the conductors inside cable 125, which connects the test unit to the bedside monitor. A spring activated valve 200 is situated lateral to the balloon chamber and allows calibration fluid to pass into the balloon (or pumping) chamber 70 when elevated pressure in the infusion or calibration line 202 pushes the piston 205 of the valve distally. Fluid then passes through a slit 210 connecting valve 200 with balloon chamber 70 and exits the device through catheter 45.

FIG. 5 shows the actual size of the test unit when viewed from the top. Its small size allows it to be worn comfortably on the chest near a central venous catheter or on an extremity near an arterial line or peripheral venous catheter.

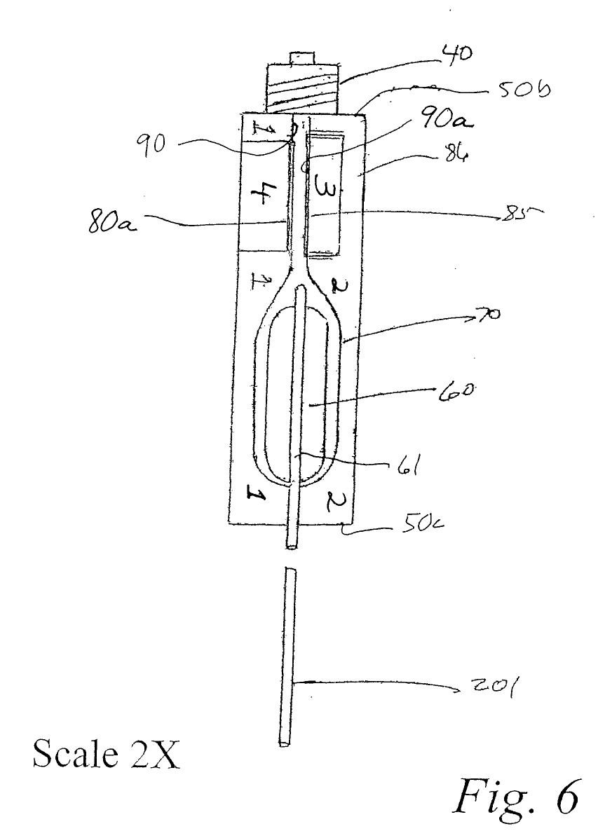

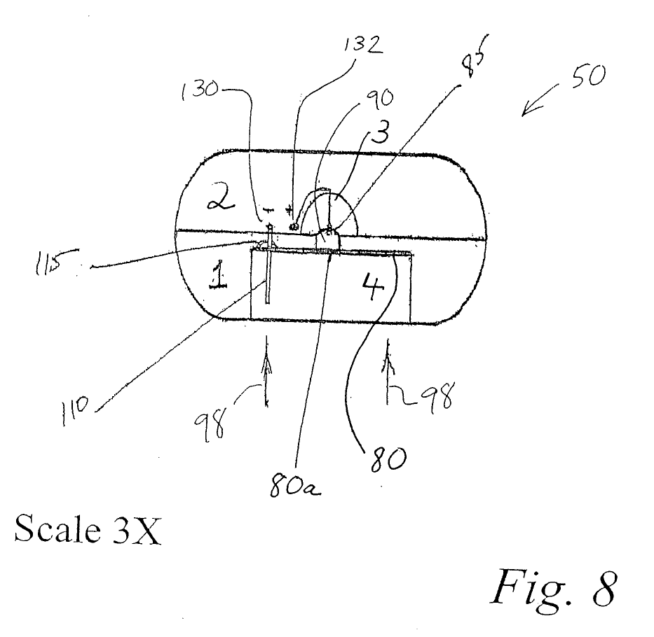

FIG. 6 is a longitudinal view along a vertical central plane of the test unit. The view shows the method of constructing the test unit from four molded plastic parts with space for the balloon chamber 70 and the passageway 90 for blood or fluid. Passageway 90 is formed in the body of test unit 50, and extends from pumping chamber 70 to the proximal end 50b of test unit 50, and provides fluid communication between pumping chamber 70 and catheter 45. Part 4 is a plastic block which is inserted from below in a final step of the assembly process. Part 4 carries the platinized kapton film 80 which is treated with glucose oxidase. The central portion 80a of film 80 forms the working electrode. The preferred method of fabricating the working electrode is as follows: the substrate is 3 layers, including a kapton plastic base, vapor coated with a smooth layer of titanium and overcoated with a smooth layer of platinum. The substrate material is cut into rectangular segments. A 0.5 to 3.0% glucose oxidase solution is formed, and cross-linked with 1 to 4% glutaraldehyde. The reaction is stopped by adding 1 to 3% bovine serum albumen. The solution is applied by pipette to the cut substrate segments, and the coated segments heated in an oven at about 50 degrees C. for 20-45 minutes. After drying, at least one coating (and preferably two or more coatings) of 2.5% AQ in water (Eastman Chemical Co.) with about 20% ethanol is applied to the glucose oxidase surface. The segments are then ready for use as the working electrode. The pure silver wire counter electrode is anodized to convert the wire surface to Ag/AgCl, as is known in the art. Part 4 is held in place after insertion by waterproof epoxy. Part 3, best seen in FIG. 8, forms the channel roof and carries a treated 300 micron silver wire 85 which comprises the counter electrode. Silver wire 85 forms a loop which is twisted together at 86, at top of part 3. Lead-out wires 130 and 132 (FIG. 8) emerge from holes in part 3 and reach the central plane of the device to exit proximally through the multi-lumen cable 125 to the bedside monitor.

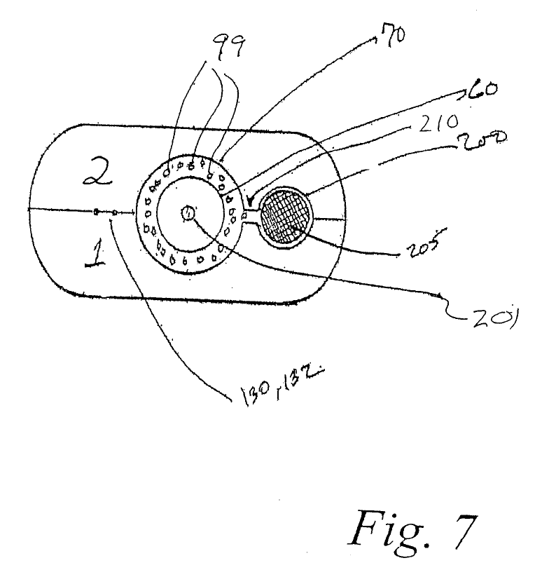

FIG. 7 is a cross-sectional view through the proximal part of the sensing unit. Parts 1 and 2 form the bottom and top halves of the unit. The two halves are best united with water-proof epoxy. The cross-sectional view shows the balloon chamber 70 with a partially inflated balloon 60 encircled by fluid containing blood cells 99. Wires 130 and 132 from the working and counter electrodes travel to the exit cable 125 between parts 1 and 2. The spring activated valve 200 is connected to the balloon chamber by a small slit 210. In the view shown, the valve is closed and the solid piston 205 blocks the passageway into the balloon chamber 70.

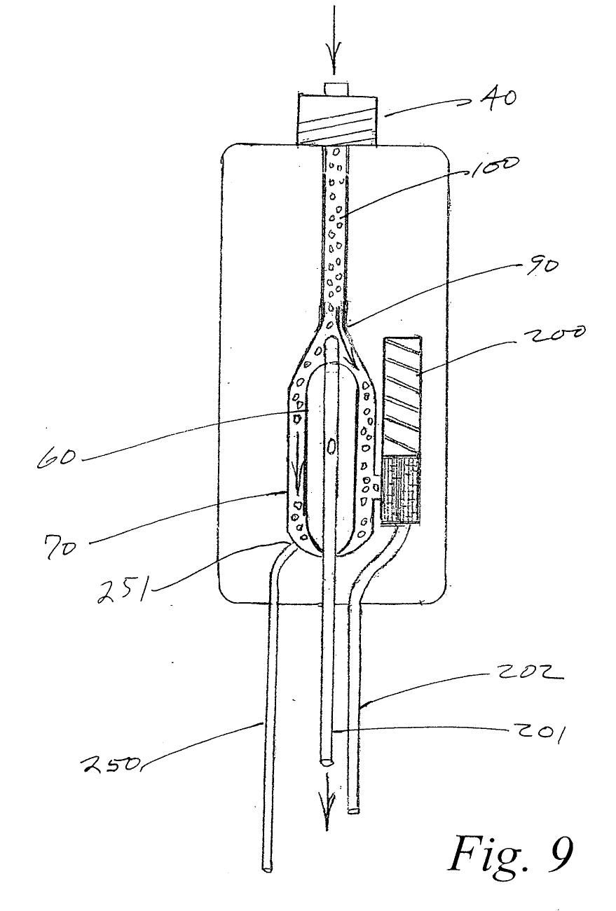

FIG. 8 is a cross-sectional view through the distal half of the sensing unit 50. This view also shows the method of electrical connection to the working electrode 80a. As shown in FIG. 9, the central portion 80a of platinized film 80 carried by part 4 acts as the working electrode 80a and forms the floor of fluid channel 90. Arrows 98 (FIG. 8) show how in production part 4, carrying the working electrode 80a, is pushed upward into the space provided in the test unit body to form the floor of the fluid channel 90.

FIG. 9 shows how a specially treated optical fiber 250 can be used to measure blood glucose with the present invention. The tip 251 of the fiber 250 is embedded in the wall of the balloon chamber 70, as shown, or alternately in the passageway 90 leading to the chamber 70. A fluorescent compound at the tip of the fiber returns light to the bedside monitor and the intensity of the returned light is a function of glucose levels over the end of the fiber. Such optical fibers and fluorescent compounds are described in the U.S. patents and applications mentioned previously. FIG. 9 shows the balloon deflating to create a negative pressure inside the chamber.

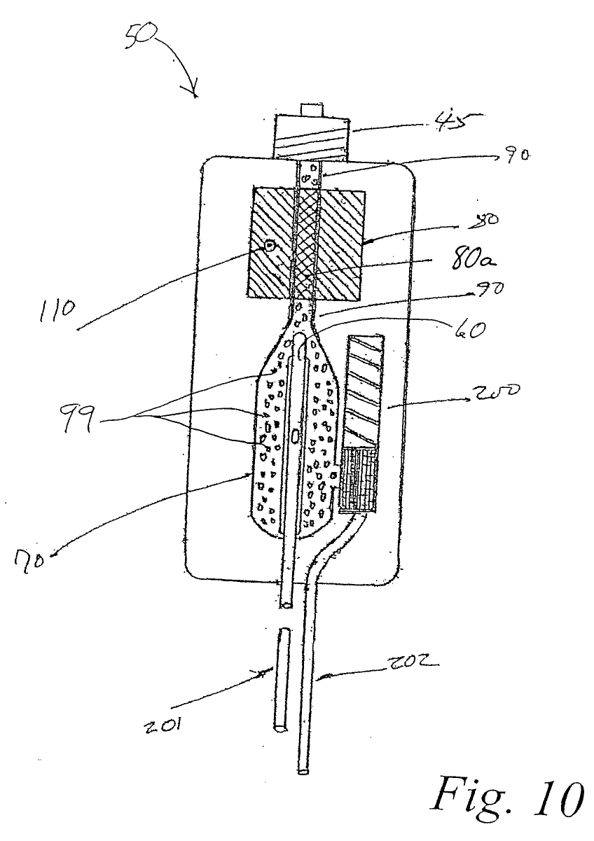

FIGS. 10-12 show the sequential steps taken during a periodic cycle of drawing blood into the test unit 50, expelling it back into the patient and then testing the small amount of residual blood remaining in the sensing unit. The unit shown uses a glucose oxidase electrode. In FIG. 10, the motor driven syringe (or actuation means) 899 inside the bedside monitor 30 is withdrawing air from the air line 201, thereby deflating the balloon (or pneumatic pumping means) 60 inside balloon chamber 70. The negative pressure created inside balloon chamber 70 brings blood from the patient's blood vessel catheter into the chamber

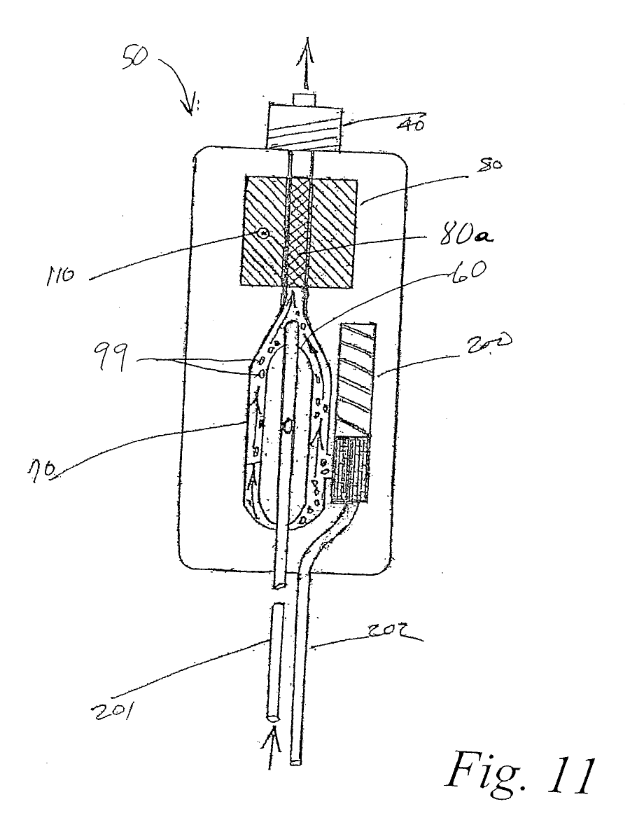

FIG. 11 shows the balloon (or pneumatic pumping means) 60 completely deflated and the chamber filled with blood 99. Balloon chamber 70 capacity is 1.8 cc. Since the closed space inside the multi-lumen central venous catheter is about 0.3 ML, and since dead space of residual blood inside the test unit is about 0.15 ML, back and forth movement of blood in and out of the balloon chamber 70 changes about 75% of the blood with each cycle.

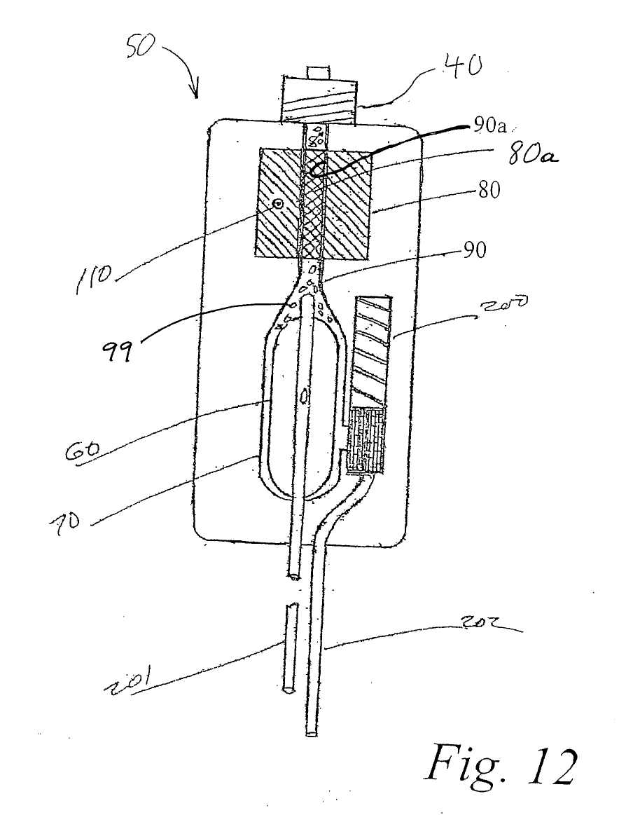

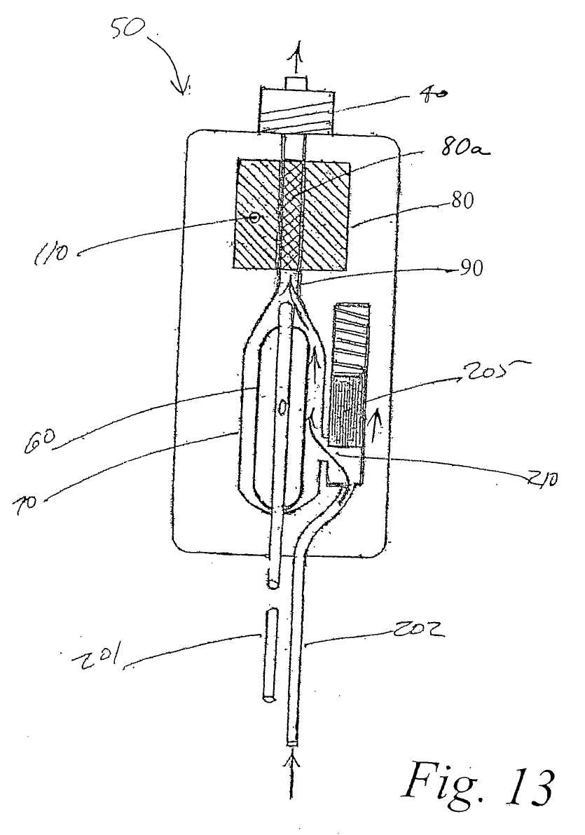

FIGS. 12 and 13 show re-inflation of the balloon 60 by the motor driven syringe (or actuation means) 899 in monitor 30 to expel blood 99 from the chamber 70 into the patient's intra-vascular catheter. The balloon 60 is inflated until it is tightly applied to the inner wall of the balloon chamber 70 as seen in FIG. 12. Chamber 70 functions as a pumping chamber, and is referred to in the claims as a pumping chamber. Normally a small amount of blood 99a, about 200 microliters, remains in passageway 90 near the working electrode 80a. The test period may last about 20 seconds, after which the balloon is deflated and the cycle is repeated again. Blood may be tested either before or after the pumping chamber is emptied. A testing area 90a is formed by the portion of channel 90 that has the working electrode 80a as its floor. The testing may be performed on the residual blood in testing area 90a as shown in FIG. 12.

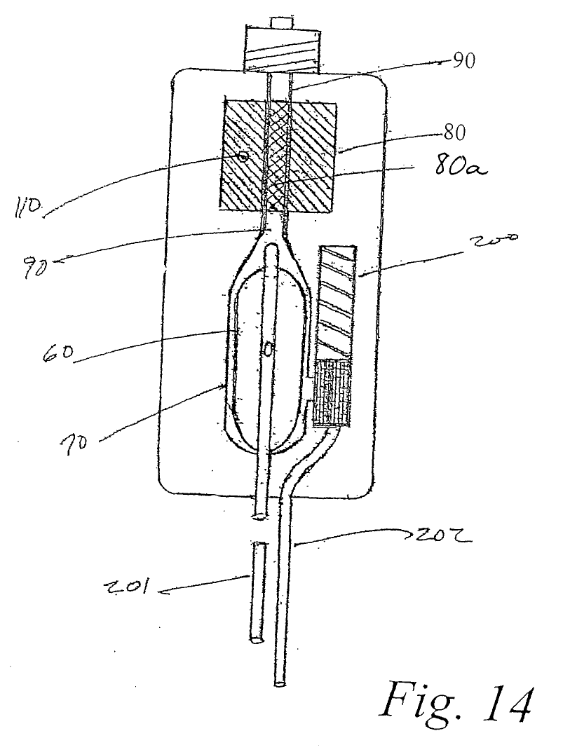

FIGS. 13 and 14 show how the pressure-activated valve 200 is used in calibration of the test unit 50. Forward movement of fluid in the calibration line 202, as shown by arrows in FIG. 13, raises the fluid pressure and causes distal movement of the valve piston 205, which exposes the passageway 210 into the balloon chamber 70. About 4-5 ML of calibration fluid flows through the balloon chamber 70, and into the patient's blood vessel catheter. The device is then allowed to remain undisturbed during calibration as shown in FIG. 14. Following calibration, the automatic periodic testing for blood glucose is begun again.

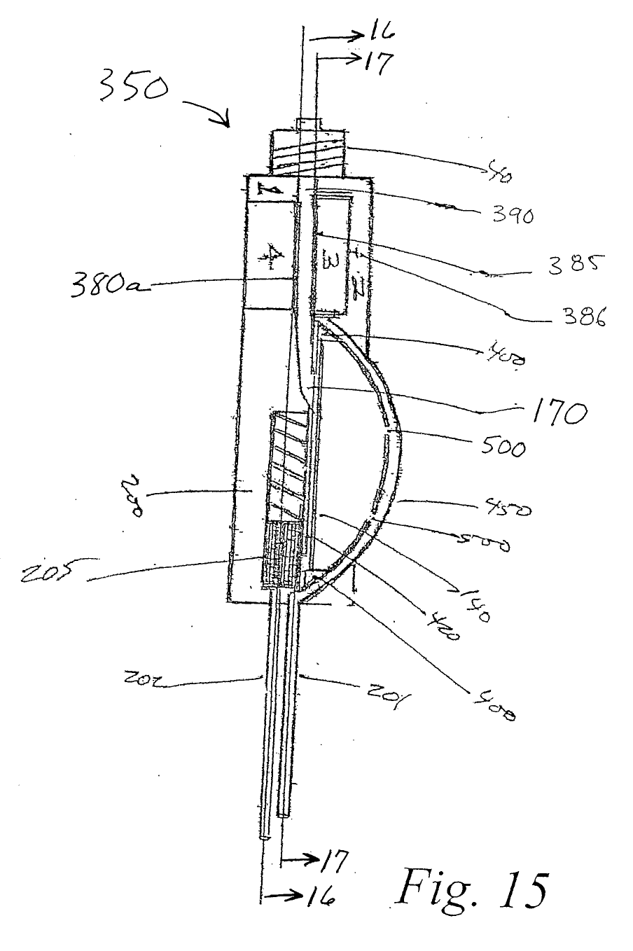

FIG. 15 is a longitudinal cross-sectional vertical view thru the central plane of the elastic membrane version 350 enlarged 2× for clarity. Test unit 350 is approximately 57 mm long, 3 mm wide and 15 mm high except through the proximal half of the device where a dome 450 increases the total height to about 21 mm. The elastic membrane 140 is stretched across the floor of the dome and is attached to an internal ring 400. Either a latex or polyurethane membrane is suitable for this application. A thin floor 420 with openings for blood and calibration fluid lies under the elastic membrane and rests against it until the membrane is drawn upward by negative pressure. A fluid channel 390 leads from a luer fitting 340 to an opening 170 in the floor of the dome, under the elastic membrane. Working and counter electrodes 390a and 385 are in the bottom and top of the fluid channel and are similar to those seen in FIGS. 4 and 5. A spring-activated valve 200 allows calibration fluid under pressure to open the valve and enter the test unit whenever calibration is required.

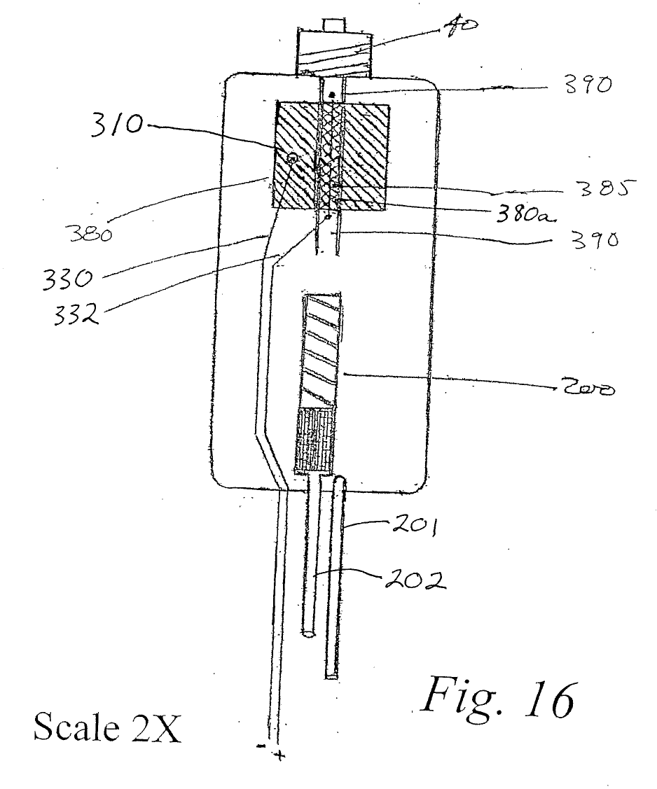

FIG. 16 is a cross-sectional horizontal view along the plane 16-16 of FIG. 15. A square piece of platinized kapton 380 may be used to form the floor of the fluid channel 390. Fluid passes over only the central 2.5 mm of platinized surface 380, which is the working electrode 380a. Electrical connection to the working electrode 380a is made with a metal pin 310, which perforates the platinized kapton near the edge of the film. A solid electrical connection between the pin and platinized film can be made with conductive epoxy. Electrical wires 330 and 332 lead from the working and counter electrodes 380a and 385 to the cable connecting the test unit to the bedside monitor. The spring-activated valve 200 is below the dome floor and opens when calibration fluid under pressure moves the valve piston and exposes the small opening into the dome.

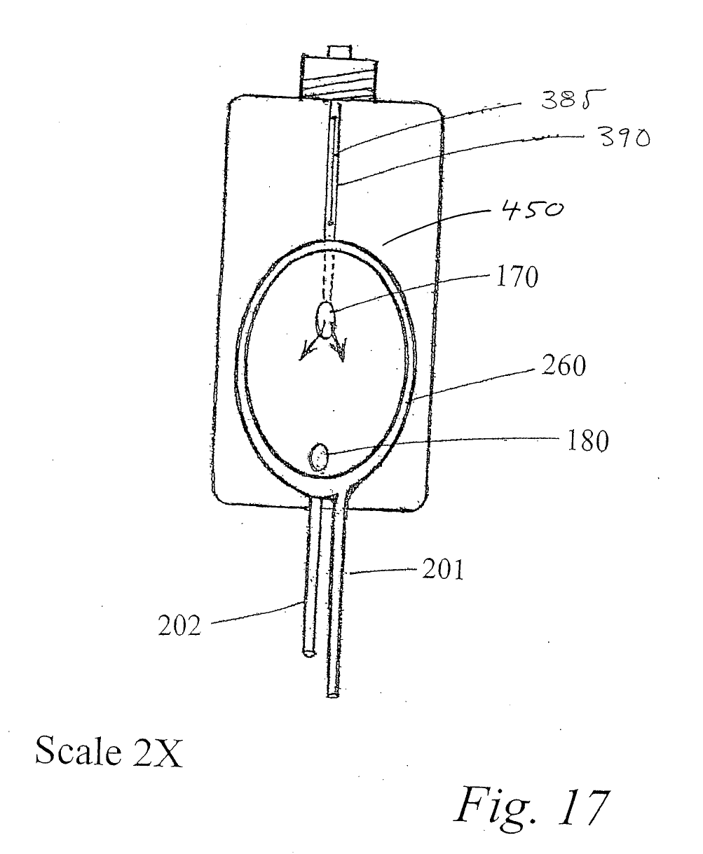

FIG. 17 is a horizontal cross-sectional view along the plane of axis 17-17 of FIG. 15. The foot print of the dome 450 is seen and the two openings into the floor of the dome. One opening distally 170 is connected to the fluid channel from the patient, and one opening proximally 180 connects to the calibration fluid line. The counter electrode 385 is seen in the narrow, top part of the fluid channel. The dome is hollow, being comprised of two parts separated by a 2 mm air space. The air line 201 leading from the bedside monitor connects with the hollow space 260 between the inner and outer walls of the dome, allowing air to be withdrawn from the dome through perforations 500 in the inner wall. Air can be later returned through the air line to restore normal air pressure under the dome.

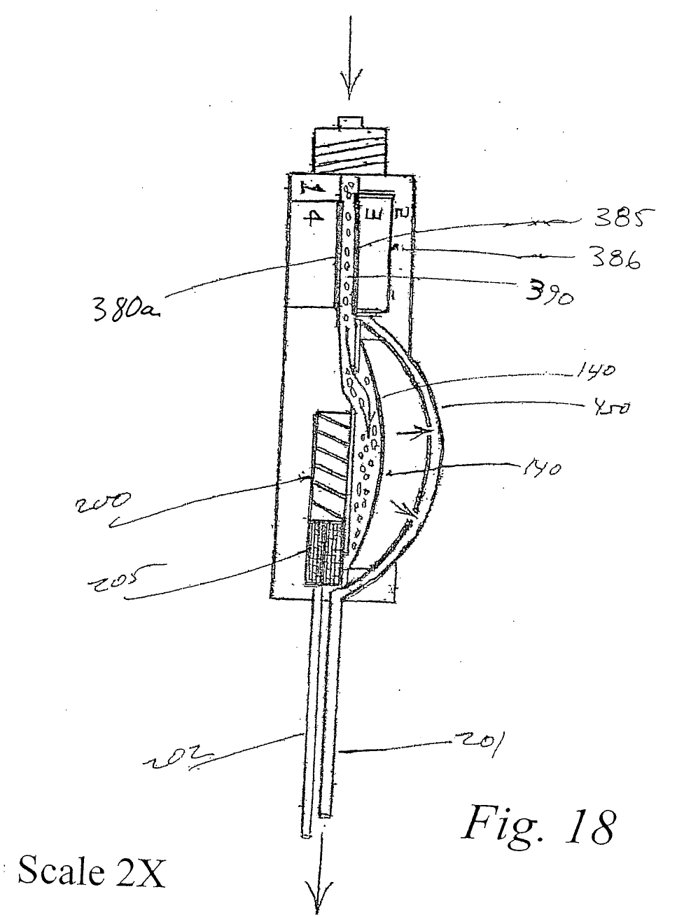

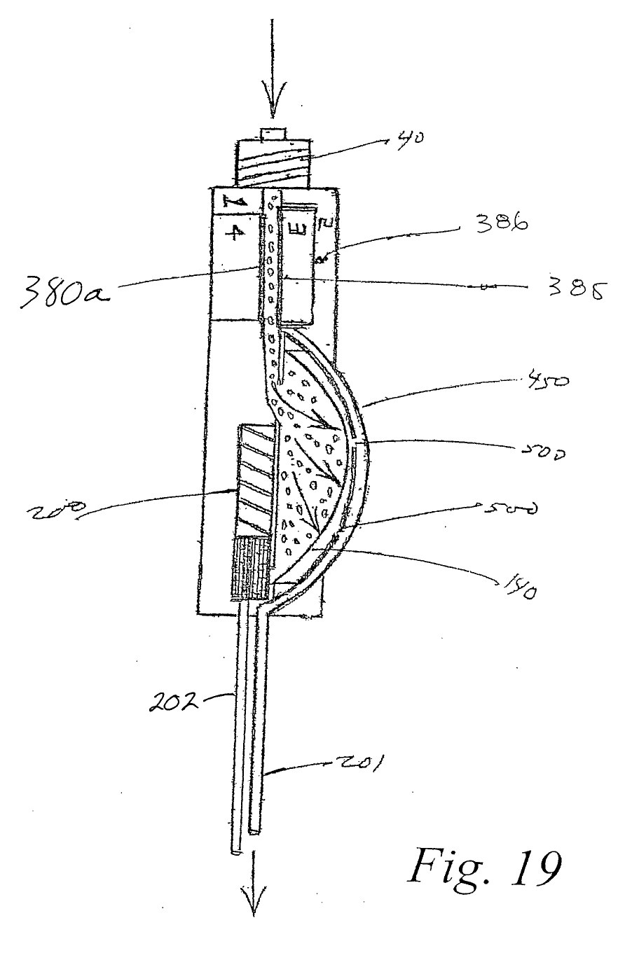

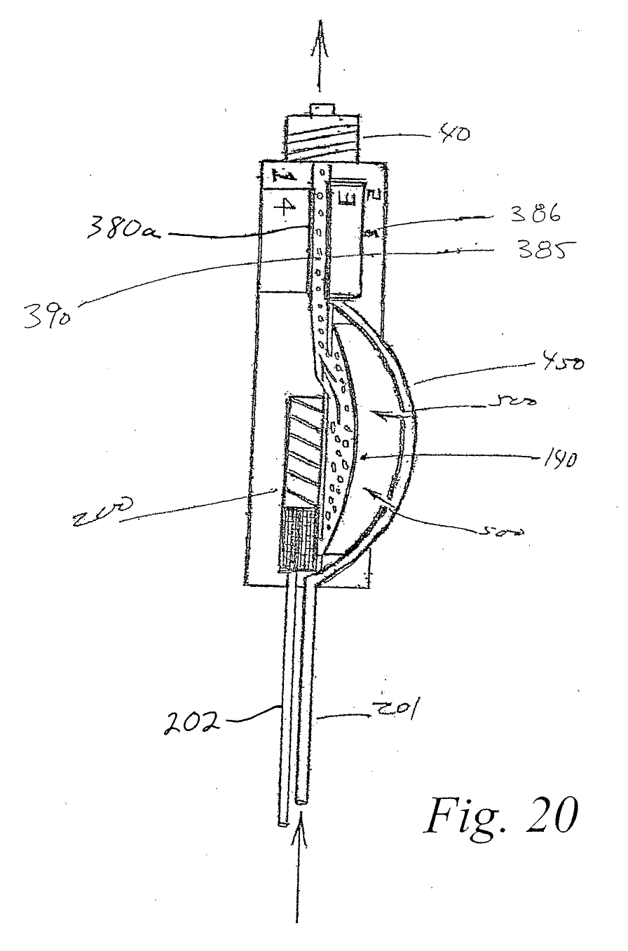

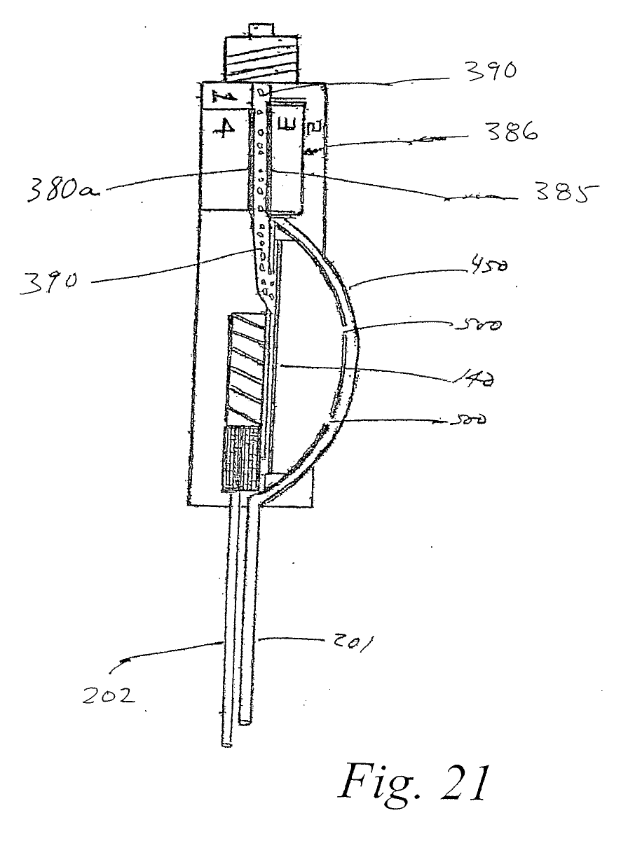

FIGS. 18-21 show the sequence of steps in measuring blood glucose. In FIG. 18, air is withdrawn from the dome 450 of the test unit via the airline 201 connected to a precision motor driven syringe 899 inside the monitor. As air is withdrawn, the elastic membrane (or pneumatic pumping means) 140 is drawn upwards causing a negative pressure below it. Dome 450 functions as a pumping chamber, and is referred to in the claims as a pumping chamber. The negative pressure created under the membrane draws in blood from the patient's blood vessel catheter. The membrane is approximated to the inner wall of the dome (FIG. 19) and the space under the elastic membrane will then contain just under 2 ML of blood. The motor driven syringe is now reversed (FIG. 20) and normal air pressure is restored under the dome, forcing most of the blood under the elastic membrane back into the patient. As shown in FIG. 21, a test is done on the residual blood in the fluid channel 390 between the working and counter electrodes. The test chamber 390a is the portion of fluid channel 390 that has the working electrode 380a as its floor. The blood glucose test may also be done before expelling the sample back into the patient's circulation. The test cycle is repeated about every 60 seconds. The method of calibrating the test unit by forward pumping of calibration fluid through the calibration fluid line 202 by the peristaltic pump inside the bedside monitor is similar to the balloon pump version. Increased pressure inside the fluid line opens the valve 200 and allows calibration fluid of known concentration to flow over the sensing electrodes. The elastic membrane 140 is raised by slight negative pressure inside the dome. The pump is stopped and the device is calibrated. Following calibration, the normal testing cycle is repeated.

As described above, two separate means for testing the glucose level of blood are expressly disclosed. The first such “testing means” disclosed is the glucose oxidase electrode 80a with its counter electrode 85. The second “testing means” disclosed is optical fiber 250 shown in FIG. 9 with coated tip 251. Other means for testing the glucose level of blood could also be utilized without departing from the invention.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

What is claimed is:

1. Apparatus for periodically and automatically testing a patient's blood glucose level, wherein said patient has in place a venous or arterial catheter, comprising:
a disposable, wearable sensing unit having a body, said body having distal and proximal ends,
a pumping chamber formed in said body of said sensing unit,
pneumatic pumping means positioned within said pumping chamber, said pneumatic pumping means movable between first and second positions,
a passageway formed in the body of said testing unit between the pumping chamber and the proximal end of said body, said passageway providing fluid communication between the pumping chamber and said venous or arterial catheter worn by said patient,
a testing area in contact with a patient's blood which is drawn into said sensor.
actuation means for causing said pneumatic pump means to move between said first position in which said pumping chamber and said testing area are filled with blood and a second position in which said pumping chamber is emptied as blood is pumped from said pumping chamber through said passageway back into said patient, but wherein blood remains in said passageway and fills said testing area, and
means for testing the glucose level of blood in said testing area when said pneumatic pumping means is either in said first or said second position.

2. The apparatus of claim 1 wherein said glucose testing means is a glucose oxidase electrode.

3. The apparatus of claim 1 wherein said testing means is an optical fiber sensitive to the level of blood glucose.

4. The apparatus of claim 1 wherein infusion fluids are introduced either through a separate catheter or through the proximal lumen of a multi-lumen catheter, preventing the unwanted mixing of blood and infusion fluid during test cycles.

5. The apparatus of claim 1 wherein said pneumatic pumping means comprises a balloon pump.

6. The apparatus of claim 1 wherein said pneumatic pumping means comprises an elastic membrane.

7. The apparatus of claim 6 further comprising a domed surface cooperating with said elastic membrane to provide the required pumping.

8. The apparatus of claim 1 wherein about two ML of blood is withdrawn from said patient for each test cycle.

9. The apparatus of claim 1 further comprising calibration means.

10. The apparatus of claim 9 further comprising a movable piston valve.

11. The apparatus of claim 10 wherein said piston valve is actuated by fluid pressure in a calibration fluid line.

12. A method of periodically and automatically testing a patient's blood glucose level, wherein said patient is wearing a venous or arterial catheter, wherein said catheter is connected to and in fluid communication with a disposable sensing unit, and wherein said sensing unit has a pumping chamber and a pneumatic pumping means positioned in said pumping chamber, a passageway formed between said pumping chamber and the proximal end of said sensing unit, and with a testing area in the fluid path of said sensing unit, comprising the steps:
pumping blood with said pneumatic pumping means from said patient through said catheter into a pumping chamber, thereby filling said pumping chamber, said passageway and said testing area with blood,
pumping blood with said pneumatic pumping means from said pumping chamber back through said passageway and said catheter into said patient, wherein said pumping chamber is emptied but wherein said passageway and said testing area remain filled with blood, and
testing the blood glucose level of the blood in said testing area when said pumping chamber is either filled with blood or emptied of blood.

13. The method of claim 12 characterized by:
the isolation of blood samples from infusion fluid during the cycle of blood glucose testing.

14. The method of claim 12 further characterized by:
using a test cycle of sixty seconds or less to avoid the formation of clots.
